(19) United States
(12) Patent Application Publication
(10) Pub. No.: US 2007/0086976 A1
(43) Pub. Date: Apr. 19, 2007

(54) Acyclic N-halamines in antibacterial materials

(75) Inventors: Gang Sun, Davis, CA (US); Song Liu, Davis, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA (US)

(21) Appl. No.: 11/251,354

(22) Filed: Oct. 13, 2005

(51) Int. Cl. A61K 31/787; A61K 31/785; C08L 39/04

(52) U.S. Cl. 424/78.27; 424/78.3; 525/203

(57) ABSTRACT

The present invention provides biocidal polymers having N-halamine technology incorporated therein, which are useful for a wide range of applications. The microbiocidal polymer can be a homopolymer, a co-polymer or a grafted polymer. In certain aspects, the present invention provides polymers from acrylic amine-monomers such as acrylamide, which monomers readily form homopolymers, co-polymers, or a grafted polymer such as onto an existing textiles.

### BACKGROUND OF THE INVENTION

Microbial contamination on surfaces is of great concern in medical devices, health care products, water purification systems, hospitals, dental office equipment, food packaging, food storage, and the like. Consequently, biocidal materials have received much attention in recent years (See, Paulus, W. and O. Pauli., U.S. Pat. No. 3,817,702 (Jun. 18, 1974); A. J. Isquith, E. A. Abbott, and P. A. Walters., Appl. Microbial. (1972), 24(6), 859).

Among the currently investigated biocidal materials, N-halamines have been shown to provide almost instant and total kill of a wide range of microorganisms. (see, Worley, S. D. et al., Trends Polym. Sci., 11:364 (1996)). There are many advantages associated with using N-halamine structures. First, they are stable in long-term use and storage over a wide temperature range. Second, they are regenerable when activity is lost due to normal use patterns. (see, Sun, G. et al., Polymer, 37:3753 (1996); Worley, S. D. et al., The Polymeric Materials Encyclopedia, 1, A-B, p. 550 (1996); Sun, G. et al. Water Res. Bull., 1996, 32:793 (1996)). More recently, N-halamine materials have been incorporated into cellulose-containing fabrics. (see, Bickert, J. R. et al., International Conference on Safety & Protective fabric '98, 1998, p 1; Sun, G. et al., Textile Chem. Colorist, 6:26 (1998); Sun, G. et al., Textile Chem. Colorist, 31:21 (1999)). Results indicate that as little as 1% (wt) add-on of halamine structures provides powerful biocidal efficacy (6-7 log reduction) against the most common pathogens, at a contact time of two minutes.

U.S. Pat. No. 5,882,357, issued to Sun et al., on Mar. 16, 1999, discloses durable and regenerable microbiocidal textiles and methods for preparing the same. The microbiocidal textiles are prepared using a wet finishing process to covalently attach a heterocyclic N-halamine to a cellulose-based material or other polymeric material. The biocidal activity of the textiles can be regenerated by washing with a halogenated solution. In addition, U.S. Pat. No. 6,020,491, issued to Wonley et al., on Feb. 1, 2000, discloses cyclic amine monomers and polymers that are used to form biocidal N-halamine polymers. The polymers are useful as disinfectants for potable water, swimming pools, hot tubs, industrial water systems, cooling towers, air-conditioning systems, and the like.

Among many biocidal materials, heterocyclic N-halamines are advantageous in properties such as rapid kill of a broad spectrum of microorganisms, stability under repeated laundering and health and environmental safety. Polymeric biocides have been disclosed in Sun et al., Macromolecules, 2002, 35, 8909-8912 and Sun et al., J. Appl. Polm. Sci., 2001 81, 617-624.

More recently, with the advance of multiresistant pathogens as well as the threat of pathogenic microbes from terrorists, a need exists for more effective biocidal polymers. Despite the advantages of the heterocylic N-halamine technology, a need exists for newer more potent biocidal materials. The present invention provides this and other needs.

### SUMMARY OF THE INVENTION

The present invention provides biocidal polymers having N-halamine technology incorporated therein, which are useful for a wide range of applications. As such, in one embodiment, the present invention provides a microbiocidal polymer, wherein the microbiocidal polymer is a homopolymer, a co-polymer or a grafted polymer. The homopolymer comprises: a repeating unit having formula I:

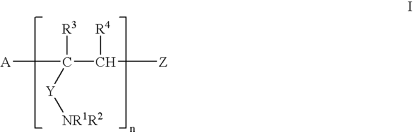

wherein:

A is selected from the group consisting of hydrogen, and optionally substituted (C₁-C₆)alkyl;

R¹ and R² are each independently selected from the group consisting of hydrogen, optionally substituted (C₁-C₆)alkyl, optionally substituted (C₂-C₆)alkenyl, and halogen, wherein at least one of R¹ and R² is halogen;

Y, if present, is a member selected from the group consisting of a carbonyl group, —NH—, an optionally substituted methylene, a optionally substituted phenylene, a divalent radical selected from the group consisting of alkylene, arylene and heteroarylene, wherein the divalent radical has at least one exocyclic amino group capable of being substituted by a halogen.

R³ and R⁴ are each independently selected from the group consisting of hydrogen, optionally substituted (C₁-C₆)alkyl and a phenyl group;

Z is a member selected from the group consisting of a bond, a hydrogen, a heteroatom with a second polymer attached thereto and a directly bound second polymer; and

n is an integer from 1 to 250 inclusive;

The co-polymer comprises: a combination of a unit of formula I and a co-monomer of formula II:

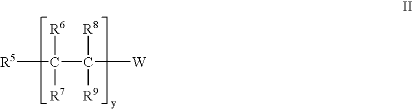

wherein:

R⁵ is a monomer of formula I, wherein Z is a bond;

R⁶, R⁷, R⁸, and R⁹ are each independently members selected from the group consisting of hydrogen, halogen, hydroxyl, cyano, (C₁-C₆)alkyl, (C₂-C₆)alkenyl, (C₁-C₆)alkoxy, (C₁-C₆)alkylcarbonyl, (C₁-C₆)alkylcarboxyl, aldehydo, amido, aryl and heterocyclyl;

W is a member selected from the group consisting of a hydrogen, a heteroatom with a second polymer attached thereto and a directly bound second polymer; and

y is an integer from 1 to 250 inclusive;

The grafted polymer comprises a second polymer with a repeating unit of formula I or a combination of formula I and formula II grafted thereto.

In certain aspects, the present invention provides polymers made from acyclic amine monomers such as acrylamide, which monomers readily form homopolymers, co-polymers, or a grafted polymer onto an existing textile (e.g., cellulose and PET). In certain other aspects, the polymers (e.g., homopolymers or co-polymers) are grafted onto an existing textile such cotton cellulose, optionally in the presence of a cross-linking agent (e.g., polyethylene glycol diacrylate (PEG-DIA)). Thereafter, the units in the polymers or grafted polymers are readily converted to N-halamine structures upon exposure to a halogenated material (e.g., chlorine bleach). The N-halamine derivatives of the corresponding polymers exhibit potent antibacterial properties against pathogenic microbes or other microorganisms e.g., Escherichia coli. Moreover, these antibacterial properties are durable and regenerable.

In another embodiment, the present invention provides methods for making a microbiocidal polymer, such as a homopolymer, a co-polymer or a grafted textile polymer.

The homopolymer prepared by a method comprising:

providing a monomeric unit having formula III admixed with an initiator,

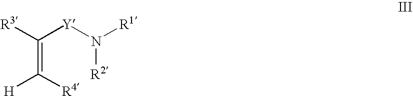

wherein:

R^{1′} and R^{2′} are each independently selected from the group consisting of hydrogen, optionally substituted (C₁-C₆)alkyl and optionally substituted (C₂-C₆)alkenyl;

Y′, if present, is a member selected from the group consisting of a carbonyl group, —NH—, an optionally substituted methylene, a optionally substituted phenylene, a divalent radical selected from the group consisting of alkylene, arylene and heteroarylene, wherein the divalent radical has at least one exocyclic amino group capable of being substituted;

R^{3′} and R^{4′} are each independently selected from the group consisting of hydrogen, optionally substituted (C₁-C₆)alkyl and a phenyl group; and

n is an integer from 1 to 250 inclusive, to generate a homopolymer; and

optionally exposing the homopolymer to a halogen source;

The co-polymer prepared by a method comprising: providing a combination of the monomer unit of formula III and a co-monomer of formula IV admixed with an initiator:

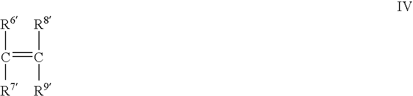

wherein:

R^{6′}, R^{7′}, R^{8′}, and R^{9′} are each independently members selected from the group consisting of hydrogen, halogen, hydroxyl, cyano, (C₁-C₆)alkyl, (C₂-C₆)alkenyl, (C₁-C₆)alkoxy, (C₁-C₆)alkylcarbonyl, (C₁-C₆)alkylcarboxyl, aldehydo, amido, aryl and heterocyclyl;

y is an integer from 1 to 250 inclusive, to generate a co-polymer; and

optionally exposing the co-polymer to a halogen source;

The grafted polymer prepared by a method comprising: providing a second polymer, a monomer of formula III or a combination of formula III and formula IV admixed with an optional crosslinker to generate a grafted polymer; and optionally exposing the grafted polymer to a halogen source.

Advantageously, the biocidal polymers of the present invention can be incorporated into medical clothing such as a surgical cover, or patient drape, or into clothing for first responders.

These and other features and advantages will become more apparent when read with the accompanying figures and detailed description that follows.

### BRIEF DESCRIPTION OF THE DRAWINGS

FIG. 1(A-B) illustrates processes of the present invention, Panel A shows one embodiment of homopolymerization and Panel B shows one embodiment of co-polymerization.

FIG. 2 illustrates one embodiment of a process of the present invention.

FIG. 3 illustrates influence of initiators on grafting acrylamide using the following reaction conditions: 2.486%, 35 mMol, a liquor ratio of 1:10, at 60° C. for 20 min, and 105° C. for 30 min.

FIG. 4 illustrates graft percentage vs. acrylamide (AM). The reaction conditions included potassium persulfate (PPS) 4.73%, 17.5 mmol, liquor ratio 1:10, 60° C. for 20 min and 105° C. for 30 min.

FIG. 5 illustrates a correlation of graft percentage based on weight increase and nitrogen analysis.

FIG. 6 illustrates the impact of temperature on one embodiment of a grafting process.

FIG. 7(A-C) illustrates the subtracted spectra of modified cotton (Panel A) AM-g-Cotton; (Panel B) AM-g-cotton bleached; and (Panel C) AM-g-Cotton Bleached+accelerated wash process.

FIG. 8 illustrates the washing durability of a grafted amide structure of the present invention.

FIG. 9 illustrates the storage stability of AM N-chloramine.

FIG. 10 illustrates the chlorine content of bleached AM-g-synthetic fabrics.

FIG. 11 illustrates the bleaching durability of t-AM-g-PET.

FIG. 12 illustrates one embodiment of pH on halogenation.

### DETAILED DESCRIPTION OF THE INVENTION

### I. DEFINITIONS

As used herein “ACS” means ammonium cerium nitrate.

As used herein “AIBN” means 2,2′-azobisisobutyronitrile.

As used herein “AN” means acrylonitrile.

As used herein “AM” means acrylamide.

As used herein “AMPDH” means 2,2′-azobis(2-methylpropionamideine.

As used herein “MMA” means methyl methacrylate.

As used herein “PPS” means potassium persulfate.

As used herein “PET” means polyethylene terephthalate (e.g., polyester).

As used herein, the term “alkyl” denotes branched or unbranched hydrocarbon chains, preferably having about 1 to about 8 carbons, such as, methyl, ethyl, n-propyl, iso-propyl, n-butyl, sec-butyl, iso-butyl, tert-butyl, octa-decyl and 2-methylpentyl. These groups can be optionally substituted with one or more functional groups which are attached commonly to such chains, such as, hydroxyl, bromo, fluoro, chloro, iodo, mercapto or thio, cyano, alkylthio, heterocyclyl, aryl, heteroaryl, carboxyl, carbalkoyl, alkyl, alkenyl, nitro, amino, alkoxyl, amido, and the like to form alkyl groups such as trifluoro methyl, 3-hydroxyhexyl, 2-carboxypropyl, 2-fluoroethyl, carboxymethyl, cyanobutyl and the like. The term “alkylene” refers to a divalent alkyl group as defined above, such as methylene(-CH₂—), propylene(-CH₂CH₂CH₂—), chloroethylene(-CHClCH₂—), 2-thiobutene-CH₂CH(SH)CH₂CH₂, 1-bromo-3-hydroxyl-4-methylpentene(-CHBrCH₂CH(OH)CH(CH₃)CH₂—), and the like.

The term “alkenyl” denotes branched or unbranched hydrocarbon chains containing one or more carbon-carbon double bonds.

The term “alkylene” refers to a divalent alkyl group as defined above, such as methylene(-CH₂—), propylene(-CH₂CH₂CH₂—), chloroethylene(-CHClCH₂—), 2-thiobutene-CH₂CH(SH)CH₂CH₂, 1-bromo-3-hydroxyl-4-methylpentene(-CHBrCH₂CH(OH)CH(CH₃)CH₂—), and the like.

The term “alkynyl” refers to branched or unbranched hydrocarbon chains containing one or more carbon-carbon triple bonds.

The terms “antimicrobial,” “microbicidal,” or “biocidal” as used herein, refer to the ability to kill at least some types of microorganisms, or to inhibit the growth or reproduction of at least some types of microorganisms. The polymers prepared in accordance with the present invention have microbicidal activity (antimicrobial) against a broad spectrum of pathogenic microorganisms. For example, if the polymer is grafted to a textile, the textiles have microbicidal activity against representative gram-positive (such as Staphylococcus aureus) and gram-negative bacteria (such as Escherichia coli). Moreover, the microbicidal activity of such textiles is readily regenerable.

The term “arylene” refers to a divalent aryl group, which can optionally be substituted. The arylene group preferably has at least one exocyclic amino group that is capable of being halogenated.

The term “halogen” means bromine, chlorine, fluorine, iodine and mixtures thereof.

The term “heteroarylene” refers to a divalent heteroaryl group that includes at least one sulfur, oxygen, or nitrogen in the aromatic ring, which can optionally be substituted. Non-limiting examples are, furylene, pyridylene, 1,2,4-thiadiazolylene, pyrimidylene, thienylene, isothiazolylene, imidazolylene, tetrazolylene, pyrazinylene, pyrimidylene, quinolylene, isoquinolylene, benzothienylene, isobenzofurylene, pyrazolylene, indolylene, purinylene, carbazolylene, benzimidazolylene, and isoxazolylene. The hetroarylene preferably has at least one exocyclic amino group that is capable of being halogenated.

### II. GENERAL

In one embodiment, the present invention provides acyclic amine monomers that can be used to form microbiocidal polymers. In certain aspects, the acyclic amine (e.g., acrylamide) monomers are homopolymerized, co-polymerized, or alternatively, grafted onto textiles, fabrics and polymers. The polymers are readily converted to N-halamine structures on exposure to a halogen source such as a halogen containing solution (e.g., commercially available chlorine bleach). The N-halamine derivatives of the corresponding polymers exhibit potent antibacterial properties against microorganisms such as Escherichia coli, and these properties are durable and regenerable.

Advantageously, the polymers, textiles, and fabrics can be made biocidal by reacting the corresponding unhalogenated polymers, textiles, and fabrics, with a halogen source. Suitable halogen sources include calcium hypochlorite, sodium hypochlorite (e.g., CLOROX®), sodium hypobromide, N-chlorosuccinimide, N-bromosuccinimide, sodium dichloroisocyanurate, trichloroisocyanuric acid, tertiary butyl hypochlorite, N-chloroacetamide, N-chloramines, N-bromamines, and the like.

### III. POLYMERS AND TEXTILES

A. Homopolymers

In certain aspects, the present invention provides biocidal homopolymers. In the homopolymer embodiments, a vinyl monomer is polymerized to generate a polymer having the same repeating subunits. For example, a monomer such as acrylamide is mixed with an initiator such as sodium persulfate, and the admixture is allowed to undergo polymerization. After the reaction is complete, the homopolymer is precipitated and the polymer is thereafter filtered. Other initiators are suitable to generate the homopolymers of the present invention. Suitable initiators include, but are not limited to, water soluble and water insoluble initiators.

As shown in scheme 1, in a preferred aspect, a polymerizable acylic amine monomer is polymerized to form a homopolymer. The starting monomeric units such as acrylamide (AM), methacrylamide (MAM), tert-butyl acrylamide (TBAM), and N-phenyl acrylamide, and the like, are used to generate the various polymers of the present invention. After exposure to a solution comprising a halogen, the resultant polymers demonstrate potent biocidal activity, are durable after repeated washings and can be recharged by additional exposures to a halogen source.

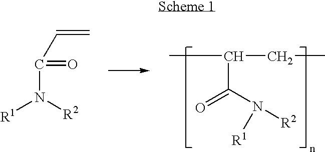

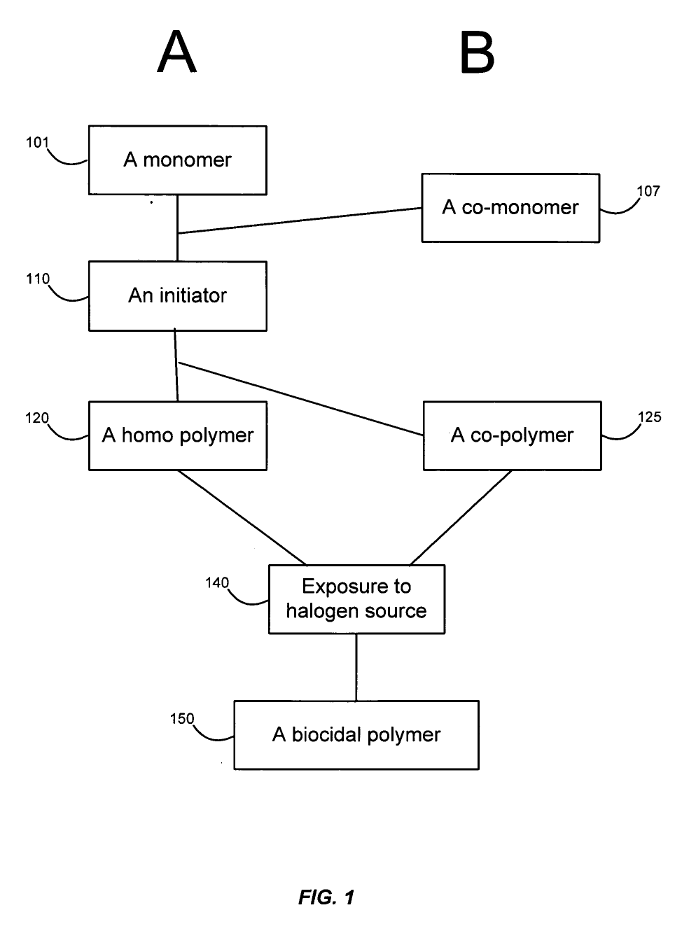

The present invention also provides a method for making a microbiocidal polymer, such as a homopolymer. As shown in FIG. 1A, the homopolymerization method includes providing a monomer 101 with an initiator 110 to form a homopolymer 120. In certain aspects, the polymerization reaction begins with the addition of the initiator to the monomer solution. The reaction is allowed to proceed and the polymer precipitates. Typical reaction conditions include an aqueous reaction solvent under inert atmosphere. The polymerization reaction is run under elevated temperature of about 75° C.-150° C., more preferably at about 90° C.-110° C. After exposure to a halogen source 140, a biocidal polymer is generated 150.

The present invention provides a homopolymer comprising: a repeating unit having formula I: 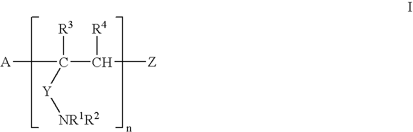
wherein: A, R¹, R², Y, R³, R⁴, Z and n have been described. In certain aspects, Y is absent (i.e., a bond). In other aspects, Y is a divalent radical such as alkylene, arylene and heteroarylene, wherein the divalent radical has at least one exocyclic amino group capable of being substituted by a halogen. In other embodiments, the divalent radical has at least two exocyclic amino groups capable of being substituted, or at least three exocyclic amino groups capable of being substituted with a halogen.

In a preferred aspect, the microbiocidal polymer of formula I has formula Ia:

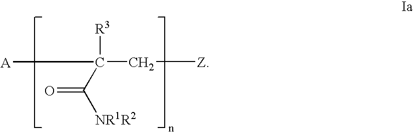

In certain aspects, R³ is hydrogen or methyl. In other aspects, R¹ and R² are both halogen. R¹ is preferably methyl or tertbutyl. In yet another aspect, formula I has formula Ib: 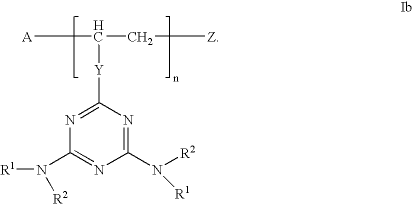
wherein: Y is a carbonyl group, —NH—, or a methylene group.

In another aspect, the present invention provides a method for making a homopolymer prepared by a method comprising: providing a monomeric unit having formula III admixed with an initiator, 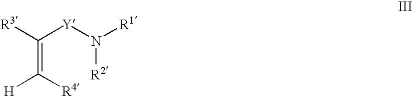
wherein: R^{1′}, R^{2′} Y′, R^{3′}, R^{4′} and n have been described, to generate a homopolymer. The method includes optionally exposing the homopolymer to a halogen source. In certain preferred aspects, at least one of R^{1′} or R^{2′} is halogenated or both are halogenated. In one aspect, formula III has formula IIIa: 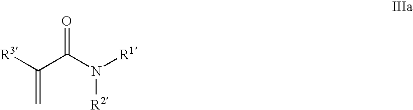
wherein R³ is hydrogen or methyl. The monomer of formula III is preferably selected from the group of acrylamide, methacrylamide, or tert-butyl acrylamide.

B. Co-Polymers

In other aspects, the present invention provides co-polymers, which can readily be made biocidal. Preferably, in the co-polymerization reactions, an acylic amine derivative such as methacrylamide, is reacted with a co-monomer such as styrene. A wide range of suitable co-monomers exist. Suitable co-monomers include, but are not limited to, acrylonitrile, styrene, acrylamide, methacrylamide, methyl methacrylate, ethylene, propylene, butylenes, butadienes and other alkenes and dienes.

The monomers of formula I can be co-polymerized with a vinyl monomer by a free radical initiation method, such as by dissolving all desired monomers in N,N-dimethylacetamide or other suitable solvent and adding, under nitrogen atmosphere, azobisisobutyronitrile, and allowing the mixture to react, at the boiling point temperature for the solvent to produce the co-polymer. The resulting unhalogenated polymers or co-polymers can then be halogenated, such as with free chlorine and bromine sources, as described herein. Additionally, the compound of formula I can be halogenated and thereafter polymerized.

In one embodiment, the present invention provides a co-polymer comprising: a combination of a unit of formula I and a co-monomer of formula II: 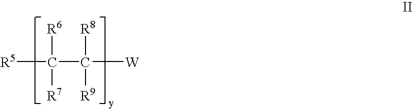
wherein: R⁵, R⁶, R⁷, R⁸, R⁹, W and y have been defined.

The concentration of the co-monomer can range anywhere from about 1% to 95% w/w or more, compared to the existing monomer. More preferably, the co-polymer monomer derivative is present in the reaction mixture at about 1% to about 30%, such as 5% to about 20%, for example, 5%, 10%, 15%, 20%, 25% or increments in-between. Similar to the homopolymerization reaction, a polymerization initiator is added such as AIBN and the co-polymerization reaction is allowed to proceed. After the co-polymerization has been effectuated, the co-polymer is precipitated and filtered. The co-polymers can thereafter be made biocidal by reacting the corresponding unhalogenated co-polymer with a halogen source.

As shown in scheme 2, in preferred aspects, a polymerizable acylic-amine monomer is polymerized with a co-monomer to form a co-polymer of the present invention. The resultant co-polymers demonstrate potent biocidal activity, are durable after repeated washings and can be recharged by additional exposures to a halogen source.

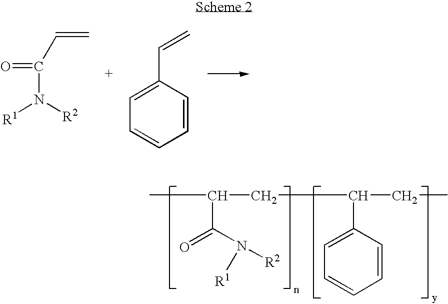

The present invention also provides a method for making a microbiocidal co-polymer. With reference to FIG. 1B, the co-polymerization method includes providing a monomer 101 with a co-monomer 107 and an initiator 110 to form a co-polymer 125. In certain aspect, the polymerization reaction begins with the addition of the initiator. The reaction is allowed to proceed and the co-polymer precipitates. Typical reaction conditions include aqueous or organic solvent under inert atmosphere. The polymerization reaction is run under elevated temperature of about 75° C.-150° C., more preferably at about 80° C.-110° C. After exposure to a halogen source 140, a biocidal polymer is generated 150.

In one aspect, the present invention provides a co-polymer prepared by a method comprising: providing a combination of the monomer of formula III and a co-monomer of formula IV admixed with an initiator: 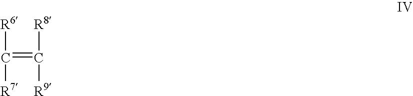
wherein: R^{6′}, R^{7′}, R^{8′}, R^{9′}, and y have been defined, to generate a co-polymer; and optionally exposing the co-polymer to a halogen source. The co-monomer of formula IV is preferably a member selected from acrylonitrile, styrene, acrylamide, methacrylamide, methyl methacrylate, and vinyl acetate.

In certain embodiments, each of end groups in the polymers are hydrogen. The co-polymers of the present invention comprise at least one unit of formula I and at least one unit of formula II. 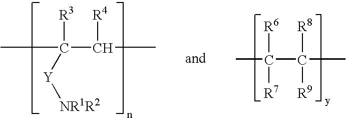
The polymers comprise varying amounts of units of “n” and “y”, i.e., the number of “n” and “y” units can be random. For example, the polymer can comprise about 10 “n” units followed by about 20 “y” units or visa versa. All combinations and variations of “n” and “y” wherein each are independently 1 to 250 are encompassed and contemplated by the present invention. Thus if the first position represents “x” and the second position “y”, such that there is a xy pair, the random variation of monomers can be 2,3; 5,10; 9,2; 1,1; 100,99; and the like. In other words, x can be any number from 1-250 and y can be any number from 1-250. In certain aspects, the units of formula I and II are grafted onto existing polymers.

C. Grafting Polymerization

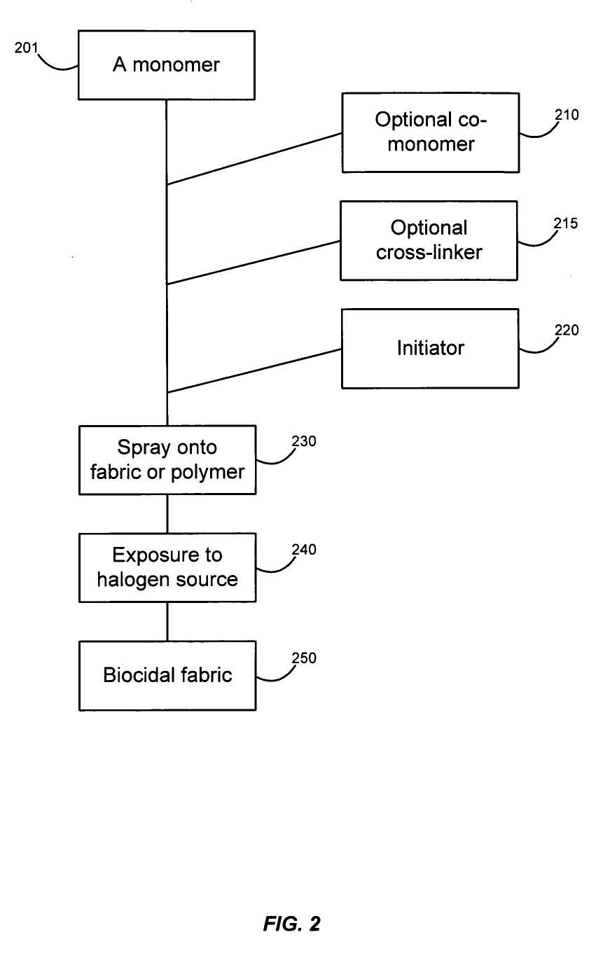

In one aspect, the present invention provides a grafted polymer comprising: a second polymer with a repeating unit of formula I or a combination of formula I and formula II grafted thereto. In yet an other embodiment, the present invention provides a graft polymerization reaction of functional monomers onto fabrics, to generate graft polymers or textiles. Preferably, the graft polymerization takes place in a pad-dry-cure process. For example, with reference to FIG. 2, finishing baths containing monomer 201, an optional co-polymer 210, an optional crosslinker 215, and initiator 220 are either padded or sprayed onto the fabric, followed by drying at elevated temperature or air drying. Preferably, the reaction occurs at an elevated temperature. With this method, the graft polymerization is favored and formation of homopolymer byproducts can be avoided or minimized. Scheme 3 shows one grafting embodiment of the present invention, wherein the second polymer is cellulose.

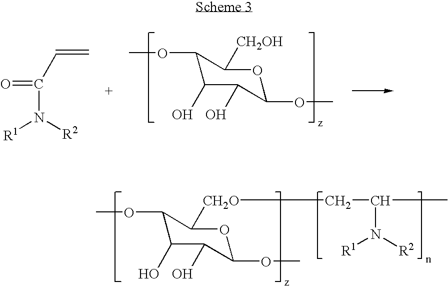

Both water-soluble and water-insoluble initiators are suitable for the polymerization reactions of the present invention. In the case of using water-insoluble initiators (e.g., BPO), all components including monomer, optional crosslinker and initiator are dissolved in an organic solvent (e.g., acetone). Then the solution can be sprayed onto the fabric. Following the air-dry of the sprayed fabric, curing at an elevated temperature can be conducted to effectuate grafting polymerization. When water-soluble initiators are used (e.g., ACS, PPS, and AMPAD), all of the chemicals (monomers and the initiator) are mixed in distilled water. Fabrics can then be dipped in a finishing bath and padded. This “dip-pad” process can optionally be repeated multiple time (e.g., twice).

In certain instances, padded fabrics are then dried. Suitable conditions include a temperature of about 40° C. to about 80° C. such as about 60° C. for 5-40 minutes (e.g., 10 min). The samples can then be cured at an elevated temperature for a certain period of time, and then washed. In one aspect, the fabric is washed according to AATCC standard 61, i.e., dried at 60° C. for 24 h, and stored in a conditioning room (e.g., 21° C., 65% RH) for over 72 h to reach constant weights.

The Percentage graft can thereafter be calculated from the relation:
Graft %=(W₂−W₁)/W₁×100 (1)
wherein W₁ and W₂ are the weights of the original and the grafted fabric, respectively.

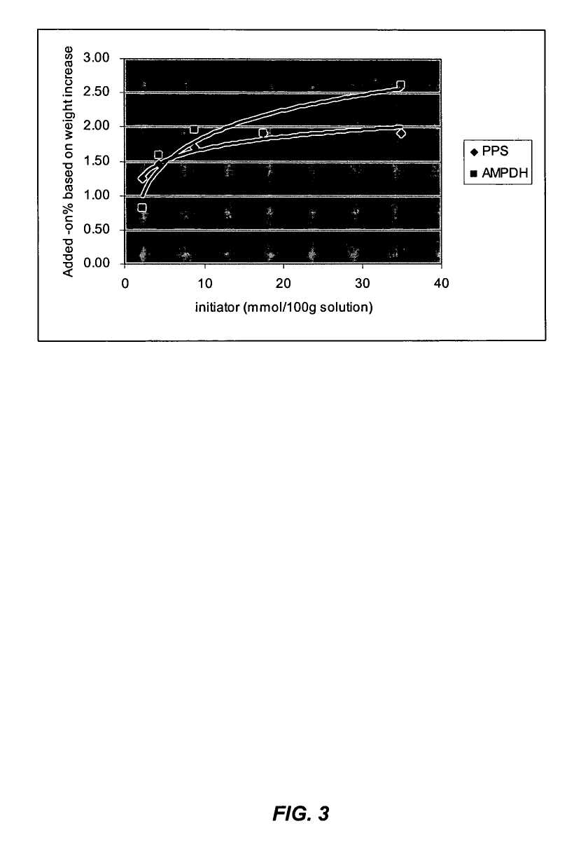

In certain aspect, in order to maximize grafting efficiency, initiators are chosen in the graft polymerization reactions that tend to have hydrogen abstraction reactions rather than radical addition, such as dibenzoyl peroxide, tert-dibutyl peroxide, tert-butyl cumyl peroxide and di-t-butylperoxyxoalate, and the like. In certain preferred aspects, 2,2′-azobis(2-methylpropionamideine) (AMPDH) or potassium persulfate (PPS) are suitable in the graft polymerization reactions of the present invention. With reference to FIG. 3, it is shown that in certain aspects, the grafting polymerization yield increases with increase of initiators concentration. The grafted polymer (e.g., textile) is thereafter cured. A wide range of curing temperatures are suitable for use in the polymerization reactions of the present invention. For example, suitable ranges include 40° C. to 160° C., more preferably, a range of 80° C.-135° C. is used.

In certain aspects, the present invention provides a grafted polymer prepared by a method comprising: providing a second polymer, a monomer of formula III or a combination of formula III and formula IV admixed with an optional crosslinker to generate a grafted polymer; and optionally exposing the grafted polymer to a halogen source. The present invention also provides the chemically modified polymer prepared by the methods described herein.

### IV. BIOCIDAL ACTIVITY

After exposure to a halogen source (e.g., sodium hypochlorite solution), the N—H bond on the grafted fabric can be transformed to an N-halamine, which provides powerful antibacterial properties.

The halogenation of the unhalogenated polymers, textiles and fabrics can be accomplished in aqueous media or in mixtures of water with common inert organic solvents such as methylene chloride, chloroform, and carbon tetrachloride, or in inert organic solvents themselves, at room temperature. Those of skill in the art will know of other solvents or solvent mixtures suitable for use in the present invention. In certain instances, the unhalogenated textiles, fabrics and polymers can be a previously-utilized acyclic N-halamine polymer that needs to be regenerated due to inactivation of the N-halamine moieties. As used herein, “halogenating” or “halogenated” polymers refers to partially as well as fully halogenated. Preferred halogens are chlorine and bromine.

For example, the polymers and grafted textiles of the present invention can be converted to N-halamines by immersing the polymers in a halogen solution (e.g., a bleach solution). In certain aspects, the halogen (e.g., chlorine) is present at about 50 ppm to about 300 ppm (e.g., 150 ppm) when cotton fabrics are used. Typically, when halogenating synthetic fabrics, the halogen is present at about 1000 ppm to about 5000 ppm (e.g., 3000 ppm). The reaction can be carried out at ambient temperature for about 30 min with stirring (e.g., bath liquor ratio was 1:50 (fabric vs finishing solution w/w), and then washed and air dried.

Various textiles and polymers can be grafted using the methods of the present invention. The second polymers suitable for use in the present invention include, but are not limited to, a plastic, a rubber, a textile material, a paint, a surface coating, an adhesives, cellulose, a polyester, wood pulp, paper and a polyester/cellulose blend. The polymeric materials suitable for the present invention include, but are not limited to, naturally occurring fibers from plants, such as cellulose, cotton, linin, hemp, jute and ramie. They include polymers from animals, based upon proteins and include, but are not limited to, wool, mohair, vicuna and silk. Textiles also include manufactured fibers based upon natural organic polymers such as, rayon, lyocell, acetate, triacetate and azlon. Textiles suitable for use in the present invention include synthetic organic polymers which include, but are not limited to, acrylic, aramid, nylon, olefin, polyester, spandex, vinyon, vinyl and graphite. Textiles also include inorganic substances such as glass, metallic and ceramic.

Various textiles are preferred to practice the invention. These include, but are not limited to, a fiber, a yarn or a natural or synthetic fabric. Various fabrics include, but are not limited to, a nylon fabric, a polyester, an acrylic fabric, NOMEX®, a triacetate, an acetate, a cotton, a wool and mixtures thereof. NOMEX is made of an aromatic polyamide material and is available from DuPont (Wilmington, Del.). NOMEX is used in fire fighting equipment.

The polymeric plastics suitable for the present invention include thermoplastic or thermosetting resins. The thermoplastics include, but are not limited to, polyethylene, polypropylene, polystyrene, and polyvinylchloride. Thermoplastics also include, polyamideimide, polyethersulfone, polyarylsulfone, polyetherimide, polyarylate, polysulfone, polycarbonate and polystyrene. Additional thermoplastics include, but are not limited to, polyetherketone, polyetheretherketone, polytetrafluoroethylene, nylon-6,6, nylon-6,12, nylon-11, nylon-12, acetal resin, polypropylene, and high and low density polyethylene.

The polymerization and chemical modification reactions can proceed by various polymerization techniques well known by those of skill in the art. For example, a free radical initiation method, a photoinitiated method or thermal initiated method are all suitable methods. For example, one can dissolve a compound of formula I in absolute ethanol or other suitable solvent and react this solution, at the boiling point for the solvent, with azobisisobutyronitrile (AIBN) under nitrogen atmosphere to produce the unhalogenated polymer.

Advantageously, the present invention provides durable and regenerable biocidal fabric, textiles and polymers. In certain preferred aspects, the structures are durable to washing and storage. For example, the grafted AM structure is durable to washing because the charged chlorine does not decrease even after 25 times washing.

Numerous applications for the biocidal polymers of the present invention exist. For instance, the biocidal polymers can provide biocidal protective clothing to personnel in the medical area as well as in the related healthcare and hygiene area. The regenerable and reusable biocidal materials can replace currently used disposable, nonwoven fabrics as medical textiles, thereby significantly reducing hospital maintenance costs and disposal fees. The microbicidal properties of the polymers of the present invention can be advantageously used for women's wear, underwear, socks, and other hygienic purposes. In addition, the microbicidal properties can be imparted to carpeting materials to create odor-free and germ-free carpets. Moreover, all germ-free environments, such as required in biotechnology and pharmaceutical industry, would benefit from the use of the microbicidal textiles of the present invention to prevent any contamination from air, liquid, and solid media.

The biocidal textiles, fabrics and polymers are effective against a wide spectrum microorganisms. Such microorganisms include, for example, bacteria, protozoa, fungi, viruses and algae. Moreover, the biocidal polymers described herein can be employed in a variety of disinfecting applications, such as water purification. In addition, the acrylic N-halamines are important in controlling microbiological contamination or growth of undesirable organisms in the medical and food industries. In addition, the textiles, fabrics and polymers herein can be used as preservatives and preventatives against microbiological contamination in paints, coatings, and on surfaces.

### V. EXAMPLES

Experimental

A. Materials

Fabrics of nylon-66 #306A, polyester #755H (PET), polypropylene #983 (PP) and pure cotton print cloth #400 were purchased from TestFabrics Inc. Benzoyl peroxide (BPO, commercially available from Acros), and potassium persulfate (PPS, commercially available from Acros) were recrystallized from systems of chloroform/methanol and distilled water, respectively. Acrylamide (AM), methacrylamide (MAM), tert-butyl acrylamide (TBAM), polyethylene glycol diacrylate (PEG-DIA) and 2,2′-azobis(2-methylpropionamideine) (AMPDH) were purchased from Aldrich and used as received.

B. Instrumentation

FTIR spectra were taken on a Nicolet Magana IR-560 spectrometer using KBr pellets. The samples were made thin enough to ensure that the Beer-Lambert law was fulfilled.

### Example 1

A. Grafting polymerization

Both water-soluble and water-insoluble initiators were selected for the reaction, and two separate processes were employed. In the case of using water-insoluble initiators (e.g., BPO), all components including monomer, crosslinker and initiator were dissolved in acetone. The so-formed BPO acetone solution was dropped into monomer solution containing TX-100 and softener under fast stirring to form an emulsion as the finishing bath. Then the solution was sprayed onto the fabric. In case of water-soluble initiators (ACS, PPS, and AMPAD) were used, all of the chemicals (monomers and the initiator) were mixed in distilled water. Fabrics were dipped in finishing baths and padded at a required expression. This “dip-pad” process was repeated twice. Padded fabrics were dried under at 60° C. for 10 min in an oven. The samples were cured at an elevated temperature for a certain period of time, and then washed according to AATCC standard 61, dried at 60° C. for 24 h, and stored in a conditioning room (21° C., 65% RH) for over 72 h to reach constant weights.

B. Chlorination

Conversion of acyclic amine structures in the grafted samples into acrylic N-halamines was conducted by immersing in the bleach solution (150 ppm and 3000 ppm available chlorine for cotton and synthetic fabrics, respectively) at room temperature for 30 min with stirring (bath liquor ratio was 1:50), and then washed and air dried. As needed, bleaching solution pH can be adjusted to 4 to 6 with acetic acid.

C. Grafting Structure Analysis

The treated cotton fabric was acid hydrolyzed, and the treated PET was depolymerize using an amine-catalyzed base hydrolyzing method. After grafted PET was hydrolyzed, the so-formed disodium terephthalate were separated by acidification to obtain solid terephthalic acid (TPA) and ethylene glycol (EG) was recovered by salting-out technique. The grafted TPA or EG were separated from the bulk TPA or EG respectively using flash chromatography, and then were characterized by LC-ESI/MS. After grafted cotton was hydrolyzed, the solution of hydrolysis was analyzed by LC-ESI/MS.

D. Antibacterial Assessment

Samples were submitted to the ANR analytical lab of University of California, Davis for Total Kjeldahl Nitrogen analysis. The antibacterial properties of the grafted samples were examined according to a modified Test Method 100 of American Association of Textile Chemists and Colorists (AATCC) against a Gram negative bacterium Escherichia coli. The fabrics were cut into four small pieces (ca.4 cm²), and two samples of the fabrics were put together in a sterilized container, and 1 mL of an aqueous suspension containing 10⁵-10⁶ colony forming units (CFU)/mL of E. coli were placed onto the surfaces of the fabrics. The fabrics were then covered by another two portions of the identical fabrics. To ensure sufficient contact, a sterilized 50-mL beaker was placed onto the top of the fabrics. After variable contact times, the inoculated samples were placed into 100 mL of 0.03% sodium thiosulfate aqueous solution to neutralize any active chlorine. The mixture was then vigorously shaken for 5 min. An aliquot of the solution was removed from the mixture and then serially diluted, and 100 μL of each dilution were plated onto a nutrient agar plate. The same procedure was also applied to the unhalogenated samples as controls. Viable bacterial colonies on the agar plates were counted after incubation at 37° C. for 24 h. Bacterial reduction is reported according to the following equation.
Reduction rate in numbers of bacteria (%)=(A−B)/A×100 (2)
Where A is the number of bacteria counted from untreated fabrics, and B is the number of bacteria counted from treated fabrics.

E. Influence of Initiators on Grafting

In order to maximize the grafting efficiency, those initiators which tend to have hydrogen abstraction reaction rather than radical addition were chosen. In consideration of environmental benefit, however, water soluble initiators have the priority to be adopted. Here, 2,2′-azobis(2-methylpropionamideine) (AMPDH) and potassium persulfate (PPS) were studied in the graft polymerization.

It should be mentioned that when the ratio of initiator vs. monomer was above ¼, the grafting yields of monomers reached to about 52-81%, which was much higher than those obtained in the solution grafting polymerization (below 50%). Under this reaction condition, the amount of homopolymer byproduct can be reduced dramatically.

F. Influence of Monomers on Grafting

As shown in Table 1, using the same initiator concentration, the MAM showed higher grafting yields on cotton fabrics than AM except when the PPS concentration was raised to 35 mMol/100 g. Assuming the initiator created same amount of macromolecular radicals in substrate, the higher yields may attribute to the longer grafting chain.

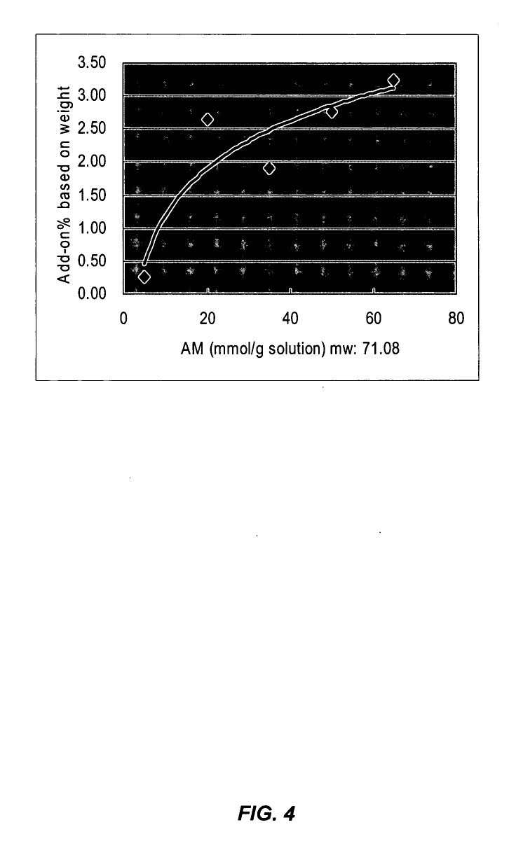

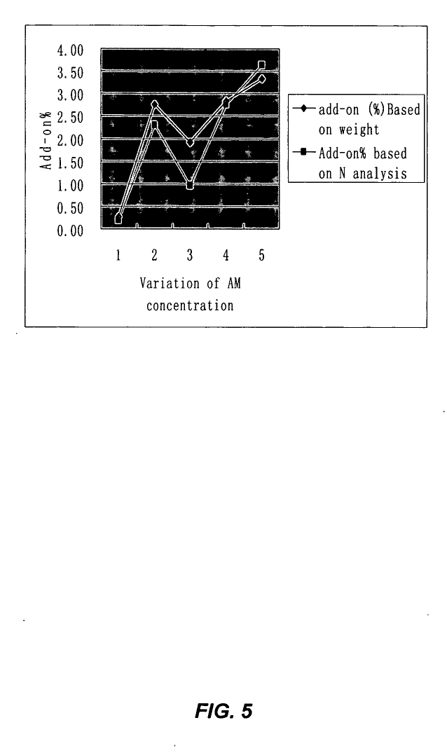

The impact of monomer concentration variation was explored in a range of 5-65 mMol/100 g, and the results are shown in FIG. 4. Within this range of monomer concentrations, grafting percentage showed steady increase, which denotes growing grafting chain size. Measurements of both weight increases of grafting reaction and nitrogen contents of the grafted fabrics were employed in characterization of the fabrics. FIG. 5 shows a good agreement between the two methods.

TABLE 1
Comparison of grafting yield between MAM and AM

| | PPS | | | |
|---|---|---|---|---|
| molar ratio of | (mMol/100 g | | add-on | Grafting yield |
| PPS/monomer | solution) | Monomer* | (%) | (%) |
| 0.0625 | 2.19 | MAM | 1.32 | 41.0 |
| | | AM | 1.25 | 38.6 |
| 0.125 | 4.38 | MAM | 1.57 | 48.6 |
| | | AM | 1.45 | 45.0 |
| 0.25 | 8.75 | MAM | 1.91 | 59.0 |
| | | AM | 1.70 | 52.6 |
| 0.5 | 17.5 | MAM | | |
| | | AM | 1.91 | 59.1 |
| 1 | 35 | MAM | 1.85 | 57.2 |
| | | AM | 1.92 | 59.3 |

G. Influence of Curing Temperature and Time

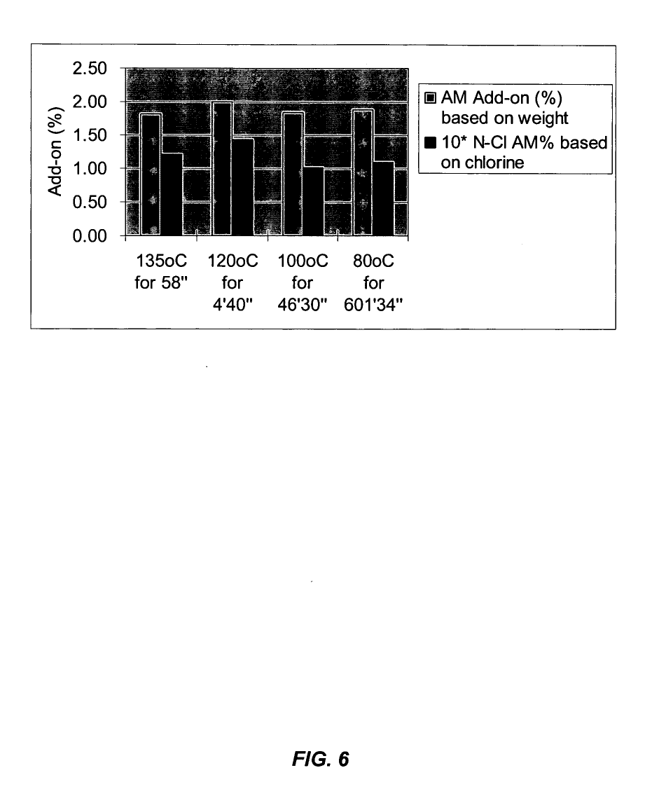

The effect of the curing temperature on the grafting polymerization was investigated over a range of 80-135° C., which was selected based on decomposition temperatures and rates of the initiators and glass transition temperatures of the polymers. Based on the activation energy and coefficient factor of the initiator PPS, half life time t_{1/2} was calculated at different temperature. The reaction durations at different temperatures were set to be 5 times t_{1/2} to ensure 97% decomposition of the initiator. The results are shown in FIG. 6.

Considering the melting point of AM is at 84.5° C., if the reaction temperature is higher than that temperature the heterogeneous reaction proceeds more effectively. However, there is not much difference of the grafting percentage in temperature range of 80-135° C. even though it reaches peak value at 120° C. Chlorination was conducted to convert the amide N—H into N—Cl for antibacterial purposes. The actual grafted AM amounts are 14-18 times more than those can be transferred to N-chlorine.

H. FTIR Study

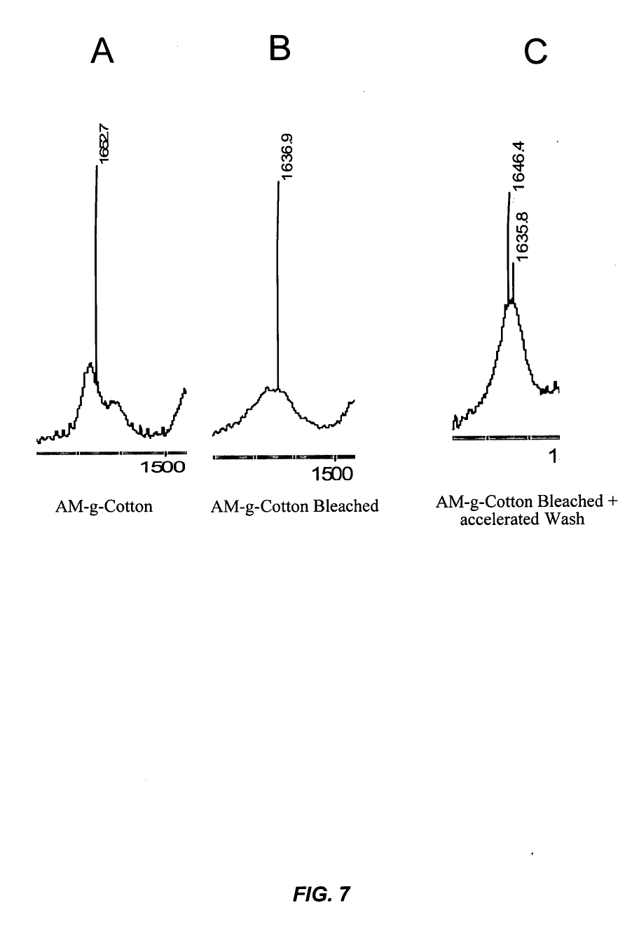

The products of the grafting polymerization were characterized by FTIR spectroscopy. With regard to FIG. 7, after subtracting the untreated cotton spectrum from grafted cotton spectrum, we can see a peak at 1652.7cm⁻¹ (Amide band due to C═O stretch) with a small should peak at 1600 cm (Amide band due to N—H bending). After bleaching, the band peak shifts to 1636.9 cm⁻¹. After following accelerated washing according to AATCC test method 61-2003, the peak shifts backwards to 1646.4 cm⁻¹, which indicates the leaching of Cl during laundering.

I. Antibacterial Assessment

After exposure to sodium hypochlorite solutions, the amide N—H bond on the grafted fabric can be transformed to N-halamine, which provides powerful antibacterial property. As an example, Table 2 compares the antibacterial capabilities of typical AM-grafted, 3-allyl-5,5-dimethylhydantoin (ADMH)-grafted and diallyl melamine-grafted cotton fabrics.

TABLE 2
Antibacterial capability Comparison of different monomer-grafted Cotton

| | Chlorine | Bacterial Number | | | | |
|---|---|---|---|---|---|---|
| | content | Part 1 in | Part 2 in | Part 3 in | Part 4 in | |
| | (ppm) | petri dish | petri dish | petri dish | petri dish | |
| ADMH-g-Cotton | 216 | 0 | 0 | 0 | 2 | |
| Other halamine-g- | 629 | 0 | 2 | 2 | 0 | 10\hat{ }6-10\hat{ }7 |
| Cotton | | | | | | reduction |
| AM-g-Cotton | 633 | 0 | 0 | 0 | 0 | of bacterial |
| (point 4 in FIG. 3) | | | | | | |
| Control (30 min | | infinite | infinite | infinite | >138 | |
| contact) | | | | | | |

J. Durability and Regenerability

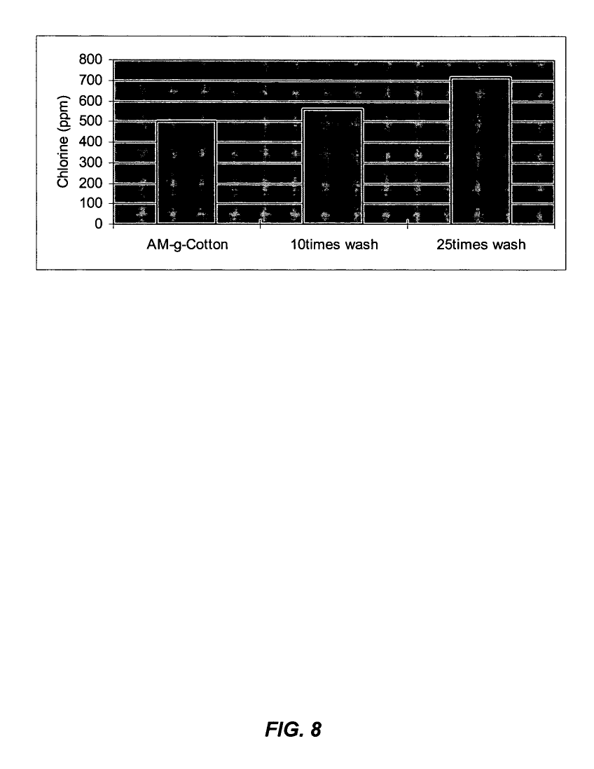

During the evaluation of durability, we had two considerations: one is the durability of the amide structure to washing and hydrolysis; another is the washing and storage durability of N—Cl bond. The AM-grafted cotton (point #4 in FIG. 5) was laundered according to AATCC test method 61-2003, followed by bleaching with sodium hypochlorite and titration. The data are shown in FIG. 8. The grafted AM structure is durable to washing because the charged chlorine did not decrease even after 25 times washing.

After the AM-g-Cotton was bleached with sodium hypochlorite, however, the total 10 nitrogen percentage dropped from 0.438 to 0.297% as shown in Table 3. There is slightly decrease of nitrogen content and relatively more loss of Cl as is seen from the increased ratio of total grafted AM vs. N—Cl transformed AM. Subsequently quench and bleach can regenerate the combine Cl 60% of original value on the AM-g-Cotton. The N-Chloramine durability and regenerability of AM-g-Cotton will fulfill certain application requirements.

TABLE 3
Stability of AM structure over bleach and wash

| | | | | | Total |
|---|---|---|---|---|---|
| | Cl | N—Cl | | Total | AM/N—Cl |
| | (ppm) | AM % | N % | AM % | AM |
| AM-g-Cotton | | | 0.438 | 2.22 | |
| AM-g-Cotton after | 550 | 0.11 | 0.297 | 1.51 | 13.9 |
| bleach | | | | | |
| 45 min accelerated | 424 | 0.09 | 0.254 | 1.29 | 15.4 |
| washing | | | | | |
| 90 min accelerated | 200 | 0.04 | 0.234 | 1.19 | 30.0 |
| washing | | | | | |
| Quenched and | 330 | 0.066 | | | |
| bleached again | | | | | |

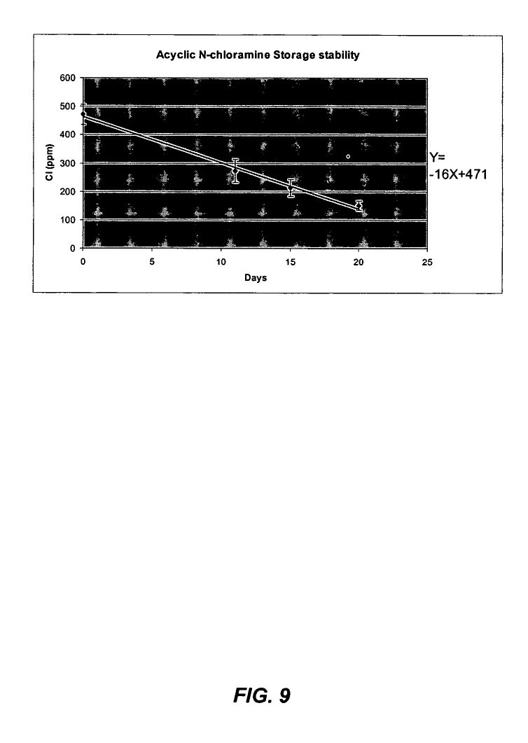

The chlorine content of the bleached AM-g-Cotton was tracked over 20 days storing in conditioning room (22° C., 65% relative humidity), and data were plotted in FIG. 9. The chlorine content fits well with a straight line dropping with a slope of 16 ppm/day.

The increase of chlorine content after regeneration can not be explained until the grafted structure is characterized by ATR/XPS or LC-MS. As shown in Table 4, the grafted t-AM structure is much more stable towards bleaching than AM. But the bulky alkyl groups also hinder the chlorination.

TABLE 4
Total nitrogen of monomer-g-PET

| | TKN % | t-AM % |
|---|---|---|
| PET blank | 0.0085 | |
| t-AM-PET bleache + 2nd washing | 0.671 | 4.14 |
| t-AM-PET bleache + 1nd washing | 0.671 | 4.14 |
| t-AM-PET bleached | 0.670 | 4.13 |
| t-AM-PET | 0.777 | 4.80 |
| AM-PET | 0.3440 | 1.70 |

K. Chlorination Rate of Grafted Rings

The grafted monomer percentage calculated from nitrogen content is more than ten times higher than that from chlorine content.

TABLE 4
Transform ratio of total nitrogen vs. chloroamine

| | Cl | Monomer % | | Monomer % | Total N/ |
|---|---|---|---|---|---|
| | (ppm) | from Cl | N % | From N | N—Cl |
| AM-g-Cotton* | | | 0.438 | 2.22 | |
| AM-g-Cotton | 550 | 0.11 | 0.297 | 1.51 | 13.9 |
| after bleach | | | | | |

*Acrylamide 2.486%, 35 mmol, PPS 4.73%, 35/2 mmol, liquor ratio 1:10

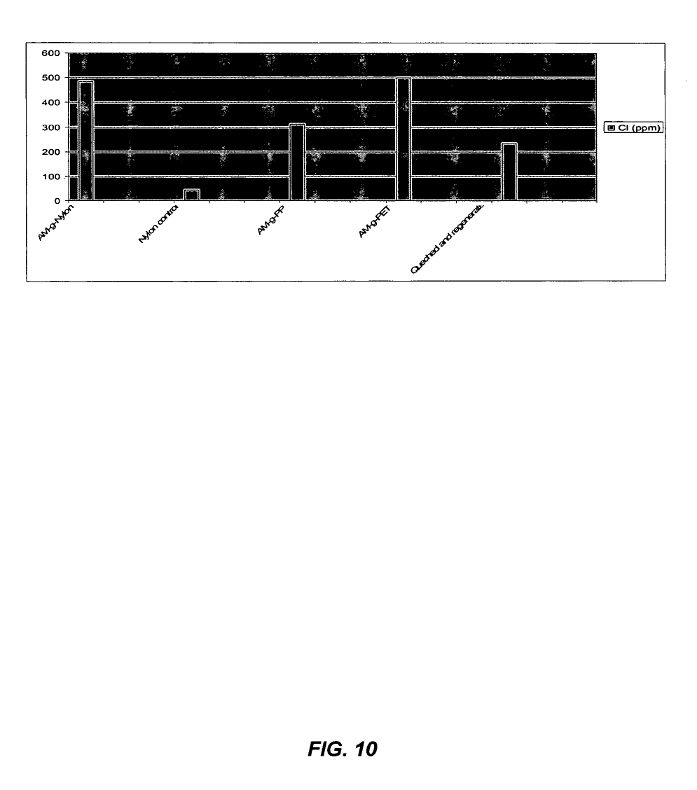

FIG. 10 illustrates the chlorine content of various embodiments of the present invention. As shown therein the amount of chlorine content for a AM-g-nylon of the present invention is about 500 ppm whereas the nylon control is about 40 ppm.

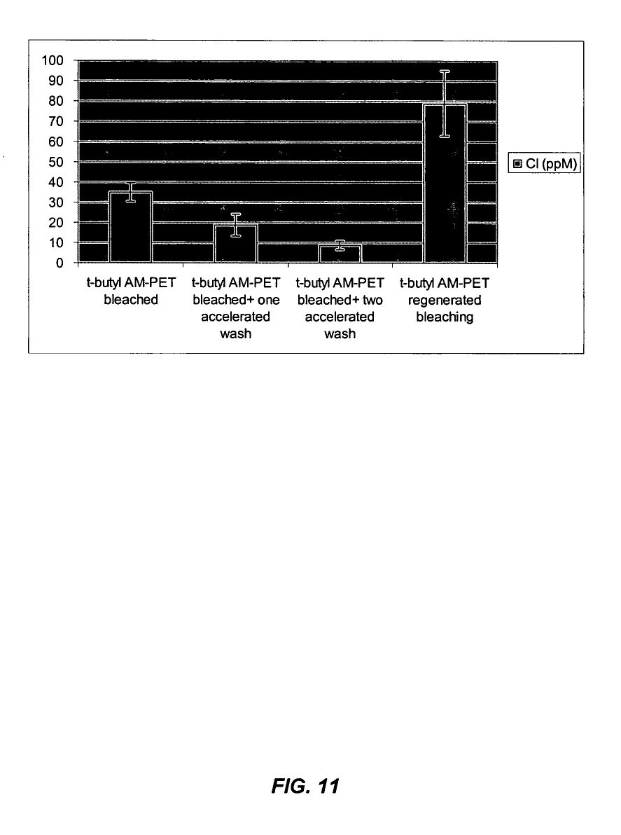

FIG. 11 illustrates the bleaching durability of t-AM-g-PET. In this embodiment, the chlorine content of the regenerated AM-polyester material was greater than the polyester treated for the first time.

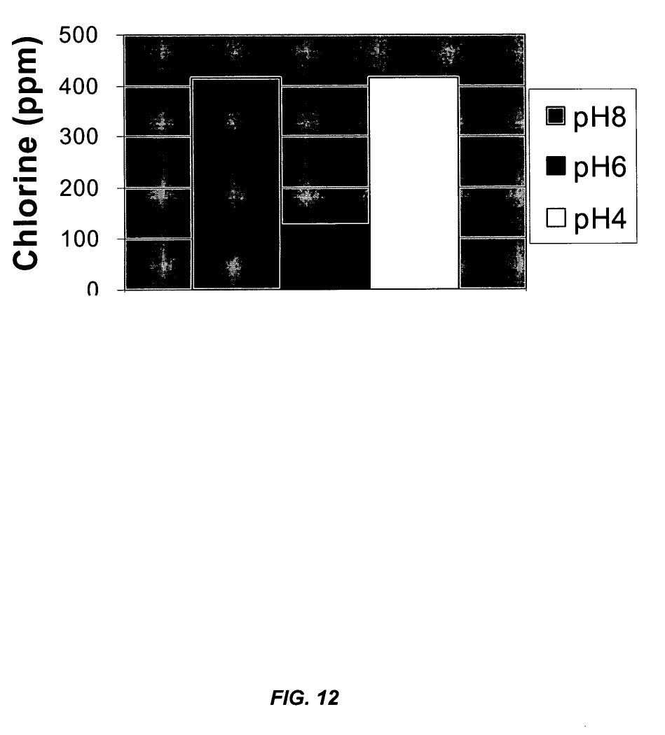

FIG. 12 shows that chlorination under pH 4 and 8 show similar efficiency, but pH 6 results in much less chlorine content being present on grafted cotton. Extending the chlorination time at pH 6 can increase the chlorine content on the same fabric. Using BPO and acrylamide or methacrylamide emulsion to finish 100% PET, shows a high amount of grafted N-chloroamine can be obtained on fabric.

### Example 2

A. This Example Illustrates Homopolymerization of Acrylamide with a Redox System in Aqueous Solution.

In a 500 ml flask equipped with a stirrer and gas inlet and outlet tube, are dissolved 2.5 g of pure acrylamide in 250 ml distilled water nitrogen is bubbled through this solution for about 10 minutes. To this solution are added 12.5 ml of 0.1 molar aqueous solutions of ferrous ammonium sulfate and 25 ml of 0.1 molar solution of hydrogen peroxide. The homopolymerization is carried out at room temperature. After about ½ h, the viscous solution is obtained. This solution is added dropwise with vigorous stirring to 2L methanol containing a few drops of hydrochloric acid. The homopolymer precipitates.

B. This Example Illustrates Polymerization of Methacrylamide with Water Soluble Initiators in Aqueous Solution

In a 250 ml three-necked flask equipped with reflux condenser and two dropping funnels 55 ml of distilled water are heated to 80° C. At this temperature, 15 g methacrylamide and a solution of 1.5 mmol potassium peroxydisulfate or 2,2′-Azobis[2-(5-methyl-2-imidazolin-2-yl)propane]di hydrochloride or 2,2′-Azobis[2-(2-imidazolin-2-yl)propane]dihydrochloride or 2,2′-Azobis(2-methylpropionamide)dihydrochloride, 2,2′-Azobis[2-(3,4,5,6-tetrahydropyrimidin-2-yl)propane]dihydrochloride in 8 ml of water are introduced dropwise into the flask under slow agitation with a magnetic stirrer. The methacrylamide polymerizes immediately as is evident from the increase in viscosity of the solution. After the reagents have been added, the temperature of reaction mixture is maintained at 80° C. for another hour. The viscous solution is mixed with a mixture of 140 ml acetone and 40 ml water, filtered and then added dropwise 1 L of a 4:1 mixture of acetone and petrol ether (50/70). The polymer precipitates and the supernatant liquid is decanted and 500 ml of acetone: petrol ether (4:1) is added. The polymer is filtered and extracted with petrol ether for 5 hours and subsequently is dried in vacuum at 50° C.

### Example 3

This Example Illustrates Graft Polymerization of Acrylamide on an Existing Polymer

Because both water-soluble and water-insoluble initiators were selected for the graft reaction, two separate ways were employed in the preparation of grafting modification baths. In the case of using water-insoluble initiators (benzoyl peroxide (BPO) and dicumyl peroxide), crosslinkers such as triallyl-1,3,5-triazine-2,4,6(1H,3H,5H)-trione, poly(ethylene glycol), the suspension agent, and the selected initiators were first mixed together, to which a certain amount of acrylamide aqueous solution was slowly added with stirring. When water-soluble initiators (Ammonium cerium (IV) nitrate, potassium peroxydisulfate, and 2,2-Azobis(2-methylpropionamidine)Dihydrochloride) were used, all of the chemicals (monomer acrylamide and the initiator) were mixed in distilled water. Fabrics were dipped in the modification baths and padded at a required expression. This “dip-pad” process was repeated twice. Padded fabrics were dried at 60° C. for 5 min in an oven followed by curing at an elevated temperature (typically 105° C. for cotton and 135° C. for polyester) for a certain period of time. Afterwards the cured fabrics were washed with a large amount of distilled water, dried at 60° C. for 2 h, and stored in a conditioning room (21° C., 65% RH) for over 72 h to reach constant weights.

### Example 4

This Example Illustrates Co-Polymerization of Acrylamide

To a 250 ml three-neck flask fitted with a mechanical stirrer and a condenser were added 55 mL of distilled water, 20 g NaCl and 1 g Na₃(PO₄). Under high speed of stirring, 0.6 g CaCl₂ in 5 mL of distilled water was added, followed by the addition of a organic phase containing 0.1 mole of styrene and acrylamide (AM) mixtures (AM was varied from 0˜40% molar ratio of the monomers), CB (0˜100% molar ratio of the total monomers), divinylbenzene (0˜6% molar ratio of the monomers) and AIBN (1% molar ratio of the monomers). The reaction temperature was then raised to 60˜65° C. and the stirring speed kept at 400 rpm during the reaction period (6 h). The polymers obtained as beads were washed thoroughly with hot water, acetone, and methanol, and dried in a vacuum at room temperature for 72 h.

The oil-in-water microemulsion was prepared with stirring on an electrical magnetic stirrer by adding styrene (4.5 wt %) to the mixture of dodecyl betaine (DB) (7.6 wt %), water, and acrylamide (2.1 wt %). The stirring speed was 1500 rpm. After being stirred for 20-30 minutes, it became transparent. The copolymerization was carried out at 70° C. by irradiation from a medium-pressure Hg lamp. The products were washed with hot water and then with toluene to remove DB and any homopolymers of acrylamide and styrene that might have formed during the reaction. The material that did not dissolve in these solvents was dried and weighed to calculate percent conversion of monomers.

An organic phase containing 0.1 mole of styrene and divinylmelamine (DVM) mixtures (DVM was varied from 0˜90% molar ratio of the monomer mixtures), chlorobenzene (CB, 50%˜150% molar ratio of the total monomers), divinyl benzene (0˜6% molar ratio of the monomers) and BPO (1% molar ratio of the monomers) was added to a 250 mL three-neck flask fitted with a mechanical stirrer and a condenser containing 60 mL of 0.6% poly(vinyl alcohol) (hydrolysis degree=88% and polymerization degree=2400) aqueous solution. The reaction temperature was then raised to 80° C. and the stirring speed kept at 150 rpm during the reaction period (6 h). The polymers obtained as beads were washed thoroughly with hot water, acetone, and methanol, and dried in a vacuum at room temperature for 72 h.

To 0.5-8 mol/L preferably 2-4 mol/L aqueous solution of II, 0.005-0.1 mol % (based on monomer) initiator such as potassium persulfate, 2,2′-Azobis[2-(5-methyl-2-imidazolin-2-yl)propane]di hydrochloride, 2,2′-Azobis[2-(2-imidazolin-2-yl)propane]dihydrochloride, 2,2′-Azobis(2-methylpropionamide)dihydrochloride, 2,2′-Azobis[2-(3,4,5,6-tetrahydropyrimidin-2-yl)propane]dihydrochloride was charged.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A microbiocidal polymer, wherein said microbiocidal polymer is a member selected from the group consisting of i) a homopolymer, ii) a co-polymer and iii) a grafted polymer;
i) said homopolymer comprising:
a repeating unit having formula I: 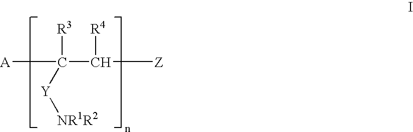
wherein:
A is selected from the group consisting of hydrogen, and optionally substituted (C₁-C₆)alkyl;
R¹ and R² are each independently selected from the group consisting of hydrogen, optionally substituted (C₁-C₆)alkyl, optionally substituted (C₂-C₆)alkenyl, and halogen, wherein at least one of R¹ and R² is halogen;
Y, if present, is a member selected from the group consisting of a carbonyl group, —NH—, an optionally substituted methylene, a optionally substituted phenylene, a divalent radical selected from the group consisting of alkylene, arylene and heteroarylene, wherein said divalent radical has at least one exocyclic amino group capable of being substituted by a halogen;
R³ and R⁴ are each independently selected from the group consisting of hydrogen, optionally substituted (C₁-C₆)alkyl and a phenyl group;
Z is a member selected from the group consisting of a bond, a hydrogen, a heteroatom with a second polymer attached thereto and a directly bound second polymer; and
n is an integer from 1 to 250 inclusive;
ii) said co-polymer comprising:
a combination of a unit of formula I and a co-monomer of formula II: 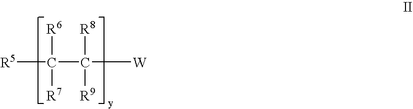
wherein:
R⁵ is a monomer of formula I, wherein Z is a bond;
R⁶, R⁷, R⁸, and R⁹ are each independently members selected from the group consisting of hydrogen, halogen, hydroxyl, cyano, (C₁-C₆)alkyl, (C₂-C₆)alkenyl, (C₁-C₆)alkoxy, (C₁-C₆)alkylcarbonyl, (C₁-C₆)alkylcarboxyl, aldehydo, amido, aryl and heterocyclyl;
W is a member selected from the group consisting of a hydrogen, a heteroatom with a second polymer attached thereto and a directly bound second polymer; and
y is an integer from 1 to 250 inclusive;
iii) said grafted polymer comprising:
a second polymer with a repeating unit of formula I or a combination of formula I and formula II grafted thereto.

2. The microbiocidal polymer of claim 1, wherein formula I has formula Ia:

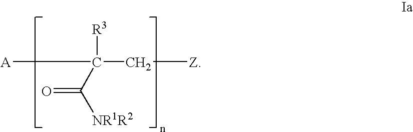

3. The microbiocidal polymer of claim 2, wherein R³ is a member selected from the group consisting of hydrogen and methyl.

4. The microbiocidal polymer of claim 2, wherein R¹ and R² are both halogen.

5. The microbiocidal polymer of claim 2, wherein R¹ is methyl.

6. The microbiocidal polymer of claim 2, wherein R¹ is tertbutyl.

7. The microbiocidal polymer of claim 1, wherein formula I has formula Ib: 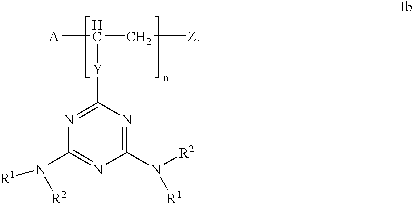
wherein:
Y is a member selected from the group consisting of a carbonyl group, —NH—, and a methylene group.

8. The microbiocidal polymer of claim 1, wherein said second polymer is a member selected from the group consisting of a plastic, a rubber, a textile material, a paint, a surface coating, an adhesive, cellulose, a polyester, wood pulp, paper, polyester and a polyester/cellulose blend.

9. The microbiocidal polymer of claim 7, wherein said textile material is a member selected from the group consisting of cellulose, a polyester, and a polyester/cellulose blend.

10. The microbiocidal polymer of claim 9, wherein said textile material is a member selected from the group consisting of fabric, yarn and fiber.

11. The microbiocidal polymer of claim 8, wherein said textile material is a member selected from the group consisting of a surgeon's gown, a cap, a mask, a surgical cover, a patient drape, a carpeting, a bedding material, an underwear, a sock and a uniform.

12. A method for making a microbiocidal polymer, wherein said microbiocidal polymer is a member selected from the group consisting of a homopolymer, a co-polymer and a grafted polymer,
i) said homopolymer prepared by a method comprising:
providing a monomeric unit having formula III admixed with an initiator, 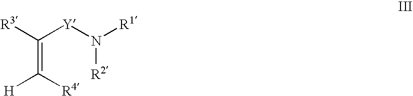
wherein:
R^{1′} and R^{2′} are each independently selected from the group consisting of hydrogen, optionally substituted (C₁-C₆)alkyl and optionally substituted (C₂-C₆)alkenyl;
Y′, if present, is a member selected from the group consisting of a carbonyl group, —NH—, an optionally substituted methylene, a optionally substituted phenylene, a divalent radical selected from the group consisting of alkylene, arylene and heteroarylene, wherein said divalent radical has at least one exocyclic amino group capable of being substituted by a halogen;
R^{3′} and R^{4′} are each independently selected from the group consisting of hydrogen, optionally substituted (C₁-C₆)alkyl and a phenyl group; and
n is an integer from 1 to 250 inclusive, to generate a homopolymer; and
optionally exposing said homopolymer to a halogen source;
ii) said co-polymer prepared by a method comprising:
providing a combination of said monomer unit of formula III and a co-monomer of formula IV admixed with an initiator: 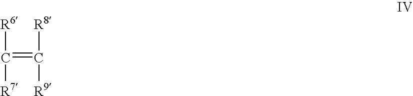
wherein:
R^{6′}, R^{7′}, R^{8′}, and R^{9′} are each independently members selected from the group consisting of hydrogen, halogen, hydroxyl, cyano, (C₁-C₆)alkyl, (C₂-C₆)alkenyl, (C₁-C₆)alkoxy, (C₁-C₆)alkylcarbonyl, (C₁-C₆)alkylcarboxyl, aldehydo, amido, aryl and heterocyclyl;
y is an integer from 1 to 250 inclusive, to generate a co-polymer; and
optionally exposing said co-polymer to a halogen source;
iii) said grafted polymer prepared by a method comprising:
providing a second polymer, a monomer of formula III or a combination of formula III and formula IV admixed with an optional crosslinker to generate a grafted polymer; and
optionally exposing said grafted polymer to a halogen source.

13. The method of claim 12, wherein formula III has formula IIIa: 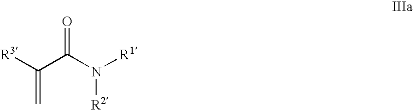
wherein R^{3′} is a member selected from the group consisting of hydrogen and methyl.

14. The method of claim 12, wherein said monomer of formula III is a member selected from the group consisting acrylamide, methacrylamide, tert-butyl acrylamide, and polyethylene glycol diacrylate.

15. The method of claim 12, wherein said co-monomer of formula IV is a member selected from the group consisting of acrylonitrile, styrene, acrylamide, methacrylamide, methyl methacrylate, and vinyl acetate.

16. The method of claim 14, wherein said co-monomer of formula IV is present in said reaction mixture in about 1 mole % to about 95 mole %.

17. The method of claim 16, wherein said co-monomer of formula IV is present in said reaction mixture in about 5 mole % to about 20 mole %.

18. The method of claim 12, further comprising treating said polymer with a solution comprising a halogen.

19. The method of claim 18, wherein said solution comprises sodium hypochlorite, sodium hypobromide, or a combination thereof.

20. The method of claim 12, wherein said second polymer is a member selected from the group consisting of a plastic, a rubber, a textile material, a paint, a surface coating, an adhesives, cellulose, a polyester, wood pulp, paper and a polyester/cellulose blend.

21. The method of claim 12, wherein said textile material is a member selected from the group consisting of cellulose, a polyester, and a polyester/cellulose blend.

22. A chemically modified polymer, said chemically modified polymer prepared by the method of claim 12.
